(19) United States
(12) Patent Application Publication
(10) Pub. No.: US 2023/0131507 A1
(43) Pub. Date: Apr. 27, 2023

(54) BIOACTIVE POLYMERS FOR OPHTHALMIC APPLICATIONS

(71) Applicant: Key Medical Technologies, Inc., San Ramon, CA (US)

(72) Inventor: Khalid Mentak, San Ramon, CA (US)

(21) Appl. No.: 18/047,865

(22) Filed: Oct. 19, 2022

Related U.S. Application Data
(60) Provisional application No. 63/271,062, filed on Oct. 22, 2021.

(51) Int. Cl. C08L 33/12; G02B 1/04

(52) U.S. Cl. CPC C08L 33/12; G02B 1/041; C08L 2203/02; C08L 2205/035; C08L 2205/02

(57) ABSTRACT

Bioactive polymers, including methods, compositions, and devices, for ophthalmic applications. The methods may include selecting a bioactive agent, conjugating the bioactive agent to a moiety to produce a bioactive monomer, and then performing a copolymerization reaction using as reactants the bioactive monomer and one or more other monomers each lacking the bioactive agent. The moiety of the bioactive monomer may react directly with at least one of the other monomers in the copolymerization reaction.

### CROSS-REFERENCE TO RELATED APPLICATIONS

This application is a non-provisional patent application of and claims priority to U.S. Provisional Patent Application No. 63/271,062 filed Oct. 22, 2021, which is incorporated herein by reference in its entirety.

### FIELD

Disclosed herein are novel materials particularly useful for ophthalmic applications and methods for making and using the same. More particularly, bioactive polymers, including methods, compositions, and devices, for ophthalmic applications are disclosed. Bioactive polymers disclosed herein are useful for producing relatively soft, optically transparent, foldable, high refractive index materials suited for use in the production of intraocular lenses, contact lenses, and other ocular implants.

### BACKGROUND

Since the 1940's optical devices in the form of intraocular lenses (IOLs) have been utilized as replacements for diseased or damaged natural ocular lenses. In most cases, an intraocular lens is implanted within an eye at the time of surgically removing the diseased or damaged natural lens, such as for example, in the case of cataracts. For decades, the preferred material for fabricating such intraocular lenses was poly(methyl methacrylate) (PMMA), which is a rigid, glassy polymer.

Softer, more flexible IOLs have gained in popularity in recent years due to their ability to be compressed, folded, rolled or otherwise deformed. Such softer IOLs may be deformed prior to insertion thereof through an incision in the cornea of an eye. Following insertion of the IOL in an eye, the IOL returns to its original, pre-folded shape due to the memory characteristics of the soft material. Softer, more flexible IOLs as just described may be implanted into an eye through an incision that is less than 4.0 mm i.e., much smaller than the 5.5 to 8.0 mm incision necessary to implant more rigid IOLs such as those made from PMMA. A larger incision is necessary for more rigid IOLs because the lens must be inserted through an incision in the cornea slightly larger than the diameter of the inflexible IOL optic portion. Accordingly, more rigid IOLs have become less popular in the market since larger incisions have occasionally been found to be associated with an increased incidence of postoperative complications, such as induced astigmatism.

In the past decade, hydrophobic polymers have been used in IOL manufacturing with some success. The ophthalmic community has accepted this type of polymer as having good physical properties and acceptable biocompatibility in ocular environments.

Polymers are very large molecules made up of many repeating monomer units. They may be natural (e.g., silk and cellulose) or synthetic (e.g., nylon and polyester). Bioactive polymers are polymers having a biological effect on a living organism, tissue, or cell. Synthetic bioactive polymers may be designed for medical applications related to the activity of a bioactive agent associated with the polymer matrix. Such applications could include ophthalmology.

Ophthalmic polymers associated with bioactive agents have not performed well clinically. The methods used to synthesize these ophthalmic polymers are very complex and result in the bioactive agent being physically located in the polymer matrix, but not covalently bound to the polymer backbone, thereby greatly reducing the bioavailability of the bioactive agent. Thus, the need still exits for foldable ophthalmic polymers that exhibit bioactive properties.

### SUMMARY

The disclosure herein provides bioactive polymers, including methods, compositions and devices, for ophthalmic applications. In one embodiment, the methods may include selecting a bioactive agent, conjugating the bioactive agent to a moiety to produce a bioactive monomer, and then performing a copolymerization reaction using as reactants the bioactive monomer and one or more other monomers each lacking the bioactive agent. The moiety of the bioactive monomer may react directly with at least one of the other monomers in the copolymerization reaction.

In one embodiment, the disclosure relates to a method of manufacturing a bioactive polymer comprising conjugating a bioactive agent to a moiety to produce a bioactive monomer; and performing a copolymerization reaction using as reactants the bioactive monomer and one or more other monomers each lacking the bioactive agent, and wherein the moiety of the bioactive monomer reacts directly with at least one of the other monomers in the copolymerization reaction.

In one embodiment, the disclosure relates to a method of manufacturing a bioactive polymer comprising conjugating a bioactive agent to a first alkenyl moiety to produce a bioactive monomer; and performing an alkenyl-based copolymerization reaction to produce the bioactive polymer, using as reactants at least the bioactive monomer and one or more other monomers each including an alkenyl moiety and lacking the bioactive agent.

In one embodiment, the bioactive agent is selected from the group consisting of: at least one of a protein, a protein fragment, a peptide, a polysaccharide, a polypeptide of at least 50 amino acids, a proteoglycan, an extracellular matrix protein, an antibacterial agent, and a therapeutic agent.

In one embodiment, the at least one of the one or more other monomers is an acrylic compound. In one embodiment, the acrylic compound is independently acrylic acid or an alpha/beta-substituted acrylic acid, an acrylate ester or an alpha/beta-substituted acrylate ester, or an acrylic amide or an alpha/beta-substituted acrylic amide.

In one embodiment, at least one monomer of the one or more other monomers includes a methacrylate compound, a methyl methacrylate compound, a hydroxyethyl acrylate compound, or a hydroxyethyl methacrylate compound. In one embodiment, the one or more other monomers include a hydrophilic monomer and a hydrophobic monomer. In one embodiment, the hydrophilic monomer has a hydroxyalkyl group bonded directly to an oxygen atom of an ester functional group. In one embodiment, the hydrophobic monomer has an alkyl group bonded directly to an oxygen atom of an ester functional group.

In one embodiment, the bioactive polymer has a refractive index of 1.4-1.6 or 1.45-1.55.

In one embodiment, the method further comprises hydrating the bioactive polymer, wherein the hydrated bioactive polymer has an equilibrium water content of 10-50% or 15-40%.

In one embodiment, the disclosure relates to a bioactive polymer produced by the methods disclosed herein.

In one embodiment, the disclosure relates to a bioactive polymer comprising a bioactive monomer constituent including a bioactive agent; and one or more other monomer constituents each lacking the bioactive agent; and wherein the bioactive polymer includes a backbone that covalently links the bioactive monomer constituent to each of the one or more other monomer constituents. In one embodiment, each unit of the bioactive monomer constituent contributes at least two carbon atoms to the backbone and each unit of each monomer constituent of the one or more other monomer constituents contributes exactly two carbon atoms to the backbone.

In one embodiment, the disclosure relates to a bioactive polymer comprising a bioactive monomer constituent including a bioactive agent; and one or more hydrophilic monomers each lacking the bioactive agent; and wherein the bioactive polymer includes a backbone that covalently links the bioactive monomer constituent to each of the one or more hydrophilic monomer constituents. In one embodiment, each unit of the bioactive monomer constituent contributes at least two carbon atoms to the backbone and each unit of each hydrophilic monomer constituent of the one or more other hydrophilic monomer constituents contributes exactly two carbon atoms to the backbone.

In one embodiment, the disclosure relates to a bioactive polymer comprising a bioactive monomer constituent including a bioactive agent; and a copolymer comprising at least two hydrophilic monomers each lacking the bioactive agent; and wherein the bioactive polymer includes a backbone that covalently links the bioactive monomer constituent to at least one of the two or more hydrophilic monomers. In one embodiment, each unit of the bioactive monomer constituent contributes at least two carbon atoms to the backbone and at least one of the two hydrophilic monomers contributes exactly two carbon atoms to the backbone.

In one embodiment, the disclosure relates to a bioactive polymer comprising a bioactive monomer constituent including a bioactive agent; and a copolymer comprising at least one hydrophilic monomer and at least one hydrophobic monomer each lacking the bioactive agent; and wherein the bioactive polymer includes a backbone that covalently links the bioactive monomer constituent to at least one of the hydrophobic monomer and the hydrophilic monomer. In one embodiment, each unit of the bioactive monomer constituent contributes at least two carbon atoms to the backbone and at least one of the hydrophilic monomer and the hydrophobic monomer contributes exactly two carbon atoms to the backbone.

In one embodiment, units of the bioactive monomer constituent are interspersed with units of the one or more other monomer constituents along the backbone.

In one embodiment, the one or more other monomer constituents form a bulk of the bioactive copolymer. In one embodiment, the one or more other monomer constituents form at least 80%, 90%, 95%, or 98% of the bioactive polymer by mass or number of units.

In one embodiment, the bioactive polymer includes an acrylic polymer

In one embodiment, the one or more other monomers includes a hydrophilic monomer. In another embodiment, the one or more other monomers includes a hydrophobic monomer. In still another embodiment, the one or more other monomers forms a copolymer that includes a hydrophilic monomer and a hydrophobic monomer. In another embodiment, the one or more other monomers may include a UV-blocking monomer, and/or a crosslinking monomer, and/or a blue light blocking monomer.

### BRIEF DESCRIPTION OF THE DRAWINGS

FIG. 1 is a non-limiting representative depiction of a reaction between a bioactive monomer having a bioactive agent and a monomer lacking the bioactive agent.

### DETAILED DESCRIPTION

### Definitions

All references to the Periodic Table of the Elements refer to the Periodic Table of the Elements published and copyrighted by CRC Press, Inc., 1990. Also, any references to a Group or Groups shall be to the Group or Groups reflected in this Periodic Table of the Elements using the IUPAC system for numbering groups. Unless stated to the contrary, implicit from the context, or customary in the art, all parts and percent are based on weight and all test methods are current as of the filing date of this disclosure. For purposes of United States patent practice, the contents of any referenced patent, patent application or publication are incorporated by reference in their entirety (or its equivalent US version is so incorporated by reference) especially with respect to the disclosure of synthetic techniques, product and processing designs, polymers, catalysts, definitions (to the extent not inconsistent with any definitions specifically provided in this disclosure), and general knowledge in the art.

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, viscosity, melt index, etc., is from 100 to 1,000, the intent is that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, the weight percent of components within compositions disclosed herein.

The term “about,” as used herein in conjunction with a numerical range, modifies that range by extending the boundaries above and below the numerical values set forth. In one embodiment, the term “about” is used herein to modify a numerical value above and below the stated value by a variance of 10%. Therefore, about 50% includes the range of 45%-55%.

As used herein, “bioactive agent” refers to substances that are capable of exerting a biological effect in vitro and/or in vivo. The bioactive agents may be neutral or positively or negatively charged. Examples of suitable bioactive agents include diagnostic agents, pharmaceuticals, drugs, synthetic organic molecules, proteins, peptides, vitamins, steroids, steroid analogs, and genetic material, including nucleosides, nucleotides and polynucleotides. In one embodiment, the bioactive agent comprises a pharmaceutical and/or drug.

As used with respect to a chemical compound, unless specifically indicated otherwise, the singular includes all isomeric forms and vice versa (for example, “hexane”, includes all isomers of hexane individually or collectively). The terms “compound” and “complex” are used interchangeably to refer to organic-, inorganic- and organometal compounds. The term, “atom” refers to the smallest constituent of an element regardless of ionic state, that is, whether or not the same bears a charge or partial charge or is bonded to another atom.

As used herein, the terms “comprising,” “including,” “having” and like terms are not intended to exclude the presence of any additional component, step or procedure, whether or not the same is specifically disclosed. In order to avoid any doubt, all processes claimed through use of the term “comprising” may include one or more additional steps, pieces of equipment or component parts, and/or materials unless stated to the contrary. In contrast, the term, “consisting essentially of” excludes from the scope of any succeeding recitation any other component, step or procedure, excepting those that are not essential to operability. The term “consisting of” excludes any component, step or procedure not specifically delineated or listed. The term “or,” unless stated otherwise, refers to the listed members individually as well as in any combination.

As used herein, the term “composition” and like terms refer to a mixture or blend of two or more components.

As used herein, the term “copolymer” refers to polymers prepared from two different monomers, and polymers prepared from more than two different monomers, e.g., terpolymers, tetrapolymers, etc.

As used herein, the term diopter (D) refers to the reciprocal of the focal length of a lens in meters. For example, a 10 D lens brings parallel rays of light to a focus at ( 1/10) meter. After a patient's natural crystalline lens has been surgically removed, surgeons usually follow a formula, based on their own personal preference, to calculate a desirable diopter power (D) for the selection of an IOL for the patient to correct the patient's preoperational refractive error. For example, a myopia patient with −10 D undergoes cataract surgery and IOL implantation; the patient can see at a distance well enough even without glasses. This is because the surgeon has taken the patient's −10 D near-sightedness into account when choosing an IOL for the patient.

As used herein, an “intraocular lens” refers to a polymeric phakic or aphakic (also referred to in the art as pseudophakic), vision-correcting device that may be implanted into a patient's eye. Phakic lenses are used to correct refractive errors such as myopia (near-sightedness), hyperopia (far-sightedness) and astigmatism (blurred vision due to poor light focusing on the retina due to an irregularly shaped cornea or, in some instances, an irregularly shaped natural lens). The natural lens remains in place when a phakic lens is implanted while the lens is removed prior to implantation of pseudophakic lens. An aphakic or pseudophakic lens is inserted in the eye subsequent to removal of the natural lens due to disease, most often a cataract; that is, clouding of the natural lens. Either type of lens may be implanted in the anterior chamber in front of the iris or in the posterior chamber behind the iris and in front of the natural lens or in the region where the natural lens was before removal. While intraocular lenses may be “hard,” that is relatively inflexible, or “soft,” i.e., relatively flexible but not foldable, for the purpose of this invention the presently preferred lens is a foldable acrylic polymer lens. A foldable lens is one that is sufficiently flexible that it can be folded into a smaller configuration to permit its implantation into the eye through a much smaller incision that is necessary for hard or soft lenses. That is, while hard and soft lenses may require a 6 mm or larger incision, a foldable lens usually requires only a 3 mm or even smaller incision. U.S. Pat. No. 7,789,509 to Mentak, U.S. Pat. No. 6,281,319 to Mentak, U.S. Pat. No. 6,635,731 to Mentak, U.S. Pat. No. 6,635,732 to Mentak, and U.S. Pat. No. 7,083,645 to Mentak, U.S. Pat. No. 7,789,509 to Mentak et al., and U.S. Pat. No. 7,399,811 also to Mentak et al. are all incorporated herein by reference in their entirety.

As used herein, “optical component,” “optical assembly” or “optical subassembly” shall mean a portion of, or a completed, ophthalmic device, assembly or subassembly. Non-limiting examples of optical components include lens bodies, optic bodies, haptics; IOL components.

As used herein, “optical polymer” refers to a polymer that is suitable for implantation into a patient's eye and that is capable of addressing ophthalmic conditions of the lens of the eye such as, without limitation, myopia, hyperopia, astigmatism and cataracts. In general such a polymer will be biocompatible, i.e., it will not cause any inflammatory, immunogenic, or toxic condition when implanted, it will form a clear, transparent, colorless (unless intentionally colored for a particular application) film-like membrane, and it will have a refractive index greater than about 1.4, preferably greater than about 1.5 and presently most preferably greater than about 1.55.

As used herein, “pharmaceutical” or “drug” refers to any therapeutic or prophylactic agent which may be used in the treatment (including the prevention, diagnosis, alleviation, or cure) of a malady, affliction, disease or injury in a patient. Therapeutically useful peptides, polypeptides and polynucleotides may be included within the meaning of the term pharmaceutical or drug.

As used herein, the term “polymer” (and like terms) is a macromolecular compound prepared by reacting (i.e., polymerizing) monomers of the same (homopolymers) or different type (copolymers). “Polymer” includes homopolymers and copolymers.

As used herein, the refractive index or index of refraction of a material is a dimensionless number that describes how light propagates through that medium. It is defined as: where c is the speed of light in vacuum and v is the phase velocity of light in the medium. For example, the refractive index of water is 1.333, meaning that light travels 1.333 times faster in vacuum than in the water.

The apparatuses and methods disclosed herein will now be described more fully hereinafter with reference to the accompanying drawings, in which embodiments of the disclosure are shown. The apparatuses and methods disclosed herein may, however, be embodied in many different forms and should not be construed as limited to the embodiments set forth herein. Rather, these embodiments are provided so that the disclosure will be thorough and complete and will fully convey the scope of the invention to those skilled in the art.

It will be appreciated by those skilled in the art that the set of features and/or capabilities may be readily adapted within the context of a standalone weapons sight, front-mount or rear-mount clip-on weapons site, and other permutations of filed deployed optical weapons sights. Further, it will be appreciated by those skilled in the art that various combinations of features and capabilities may be incorporated into add-on modules for retrofitting existing fixed or variable weapons sights of any variety.

It will be understood that when an element or layer is referred to as being “on,” “connected to” or “coupled to” another element or layer, it can be directly on, connected or coupled to the other element or layer. Alternatively, intervening elements or layers may be present. In contrast, when an element is referred to as being “directly on,” “directly connected to” or “directly coupled to” another element or layer, there are no intervening elements or layers present.

Like numbers refer to like elements throughout. As used herein, the term “and/or” includes any and all combinations of one or more of the associated listed items.

It will be understood that, although the terms first, second, etc., may be used herein to describe various elements, components, regions, and/or sections, these elements, components, regions, and/or sections should not be limited by these terms. These terms are only used to distinguish one element, component, region, or section from another element, component, region, or section. Thus, a first element, component, region, or section discussed below could be termed a second element, component, region, or section without departing from the disclosure.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90° or at other orientations) and the spatially relative descriptors used herein interpreted accordingly.

### Bioactive Polymer

In one embodiment, the disclosure provides bioactive polymers, including methods, compositions, and devices, for ophthalmic applications. In the bioactive polymers, a bioactive agent is covalently bound to the polymer backbone resulting in greater long-term stability and bioavailability. These bioactive polymers can be used to manufacture ophthalmic implants (e.g., intraocular lenses), contact lenses, and other devices.

In one embodiment, the disclosure relates to a bioactive polymer comprising a bioactive monomer constituent including a bioactive agent; and one or more other monomer constituents each lacking the bioactive agent. In one embodiment, the monomer constituent is a hydrophilic monomer. In yet another embodiment, the monomer constituent is a hydrophobic monomer. In one embodiment, the one or more other constituents for a copolymer. In one embodiment, the copolymer comprises at least two hydrophilic monomers. In another embodiment, the copolymer comprises at least two hydrophobic monomers. In still another embodiment, the copolymer comprises at least one hydrophilic monomer and at least one hydrophobic monomer.

In another embodiment, the disclosure relates to a bioactive polymer comprising a bioactive monomer constituent including a bioactive agent; and one or more other monomer constituents each lacking the bioactive agent, wherein the bioactive polymer includes a backbone that covalently links the bioactive monomer constituent to each of the one or more other monomer constituents, and wherein each unit of the bioactive monomer constituent contributes at least two carbon atoms to the backbone and each unit of each monomer constituent of the one or more other monomer constituents contributes exactly two carbon atoms to the backbone.

In another embodiment, the disclosure relates to a bioactive polymer comprising a bioactive monomer constituent including a bioactive agent; and one or more other monomer constituents each lacking the bioactive agent, wherein the bioactive polymer includes a backbone that covalently links the bioactive monomer constituent to at least one of the one or more other monomer constituents, and wherein each unit of the bioactive monomer constituent contributes at least two carbon atoms to the backbone and at least one of the one or more other monomer constituents contributes exactly two carbon atoms to the backbone.

In another embodiment, the disclosure relates to a bioactive polymer comprising a bioactive monomer constituent including a bioactive agent; and a copolymer comprising at least two other monomer constituents each lacking the bioactive agent, wherein the bioactive polymer includes a backbone that covalently links the bioactive monomer constituent to at least one of the monomer constituents of the copolymer, and wherein each unit of the bioactive monomer constituent contributes at least two carbon atoms to the backbone and at least one monomer constituent of the copolymer contributes exactly two carbon atoms to the backbone.

In another embodiment, the disclosure relates to a bioactive polymer comprising a bioactive monomer constituent including a bioactive agent and one or more hydrophilic monomers, wherein the bioactive agent is covalently linked to the one or more hydrophilic monomers.

In another embodiment, the disclosure relates to a bioactive polymer comprising a bioactive monomer constituent including a bioactive agent, one or more hydrophilic monomers, and one or more hydrophobic monomers, wherein the bioactive agent is covalently linked to at least one of the hydrophilic monomer and the hydrophobic monomer.

In another embodiment, the disclosure relates to a bioactive polymer comprising a bioactive monomer constituent including a bioactive agent, one or more hydrophilic monomers, and one or more hydrophobic monomers, wherein the bioactive agent is covalently linked to the one or more hydrophilic monomers and the one or more hydrophobic monomers.

In another embodiment, the disclosure relates to a bioactive polymer comprising a bioactive monomer constituent including a bioactive agent from about 0.001% to about 0.1% by weight of the polymer, one or more hydrophilic monomers from about 45% to 55% by weight of the of the polymer and one or more hydrophobic monomers from about 40% to 50% by weight of polymer.

In another embodiment, the disclosure relates to a bioactive polymer comprising a bioactive monomer constituent including a bioactive agent from about 0.001% to about 0.1% by weight of the polymer, one or more hydrophilic monomers from about 30% to 45% by weight of the of the polymer and one or more hydrophobic monomers from about 50% to 65% by weight of polymer.

In another embodiment, the bioactive polymer can include a cross-linking agent. In yet another embodiment, the bioactive polymer can include an ultraviolet light absorber.

### Bioactive Agent

We have surprisingly discovered that it is critical that the bioactive agent is functionalized with a group that allows concomitant polymerization with the bulk of the material. The method disclosed herein was found to improve the affinity of the bioactive agent for the polymer matrix, which in turn may reduce or eliminate phase separation, improve solubility in comonomer solutions, and/or enhance copolymerization through covalent bonding. This novel approach safeguards the specific bioactivity of the bioactive agent and affords long-term stability and optical clarity.

One important aspect of the present disclosure is the fact that a functionalized bioactive agent can be added to an existing polymer formulation without affecting the physical properties but conferring bioactivity. This results in improved biocompatibility.

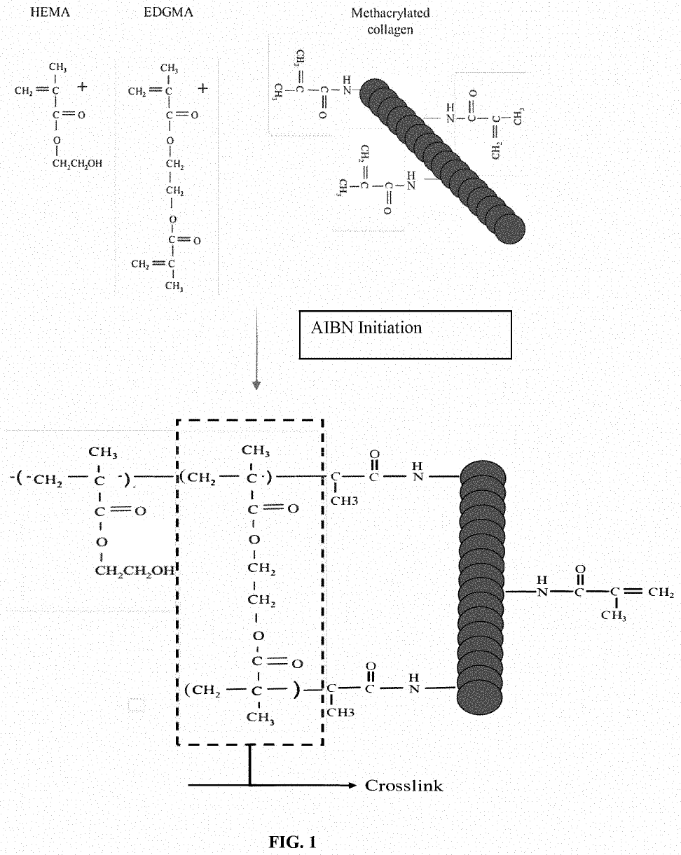

Not to be bound by a particular theory, a representative, non-limiting depiction of a method for producing a bioactive polymer is shown in FIG. 1. Methacrylated collagen is shown as the bioactive monomer with collagen as the bioactive agent. Hydroxyethyl methacrylate (HMEA) is shown as the monomer, in this case a hydrophilic monomer. HEMA and methyacrylated collagen are mixed with a crosslinker (EDGMA). To initiate polymerization, a free radical initiator (e.g., AIBN) is employed.

In one embodiment, bioactive agents can be polypeptides (e.g., proteins or protein fragments), peptides, polysaccharides, proteoglycans, antibacterials, therapeutic agents, etc). In some examples, the bioactive agent may include an extracellular matrix protein or at least a sequence of 5, 10, 25, 50, or 100 amino acids of an extracellular matrix protein. In some examples, the extracellular matrix protein may be collagen, elastin, keratin, vimentin, fibronectin, or laminin. In another embodiment, the bioactive agent can be gelation, heparin or hyaluronic acid.

In one embodiment, the bioactive agent useful in the methods and compositions disclosed herein are therapeutic substances that possess desirable therapeutic characteristics. These agents include but are not limited to: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives (including antiangiogenesis agents), anticancer chemotherapeutic agents, anti-inflammatory steroid or non-steroidal anti-inflammatory agents, immunosuppressive agents, growth hormone antagonists, growth factors, dopamine agonists, radiotherapeutic agents, peptides, proteins, enzymes, extracellular matrix components, ACE inhibitors, free radical scavengers, chelators, antioxidants, anti polymerases, antiviral agents, photodynamic therapy agents, and gene therapy agents.

In another embodiment, a wide variety of bioactive agents are available that may be suitable for use in the compositions and methods disclosed herein. The particular bioactive agent employed may vary and depends, for example, on the disease condition, the therapeutic effect being sought, the particular patient involved, and the like. Included among the bioactive agents that may be used in the compositions and methods disclosed herein are diagnostic agents, genetic materials, peptides, beta-agonists, anti-asthmatics, steroids, cholinergic agents, anti-cholinergic agents, 5-lipoxygenase inhibitors, leukotriene inhibitors, anti-neoplastic agents, antibiotics, anti-tumor drugs, radiation sensitizers, thrombolytic agents, anti-histamines, anti-coagulants, anti-inflammatories, hormones, growth factors, angiogenic factors and mitotic inhibitors. Exemplary genetic material includes, for example, nucleic acid, RNA, DNA, recombinant RNA, recombinant DNA, antisense RNA, antisense DNA, hammerhead RNA, a hammerhead ribozymes, antigene nucleic acid, ribooligonucleotides, deoxyribooligonucleotides, antisense ribooligonucleotides, and antisense deoxyribooligonucleotides.

In one embodiment, the bioactive agents are anti-neoplastic agents for the treatment of cancer. A particularly suitable anti-neoplastic agent is Taxol (Westwood Squibb). In another embodiment, the bioactive agents are thrombolytic agents for the treatment of thromboses, including thromboses in cardiac tissue. Exemplary thrombolytic agents include, for example, streptokinase, urokinase, tissue plasminogen activator, alteplase, anistreplase, reteplase and saruplase, with streptokinase being preferred.

In another embodiment, the bioactive monomer constituent including a bioactive agent is from about 0.001% to about 0.1% by weight of the composition. In another embodiment, the bioactive monomer constituent including a bioactive agent is about 0.002% by weight of the composition. In yet another embodiment, bioactive monomer constituent including a bioactive agent is from about 0.001% to about 0.005% by weight of the composition.

### Hydrophilic Monomer

Suitable hydrophilic monomers (i.e., monomers whose homopolymers are hydrophilic in accordance with this invention) include but are not limited to 2-hydroxy-ethylacrylate, 2-hydroxyethylmethacrylate, acrylamide, N-omithine acrylamide, N-(2-hydroxypropyl)acrylamide, polyethyleneglycol acrylates, polyethyleneglycol methacrylates, N-vinyl pyrolidone, N-phenylacrylamide, dimethylaminopropyl methacrylamide, acrylic acid, benzylmethacrylamide, 4-hydroxybutylmethacrylate, glycerol mono methacrylate, glycerol mono acrylate, 2-sulfoethylmethacrylate, phenoxyethyl acrylate, phenoxy ethyl methacrylate, 2-(2-ethoxyethoxy)ethyl acrylate, 2-(2-ethoxyethoxy)ethyl methacrylate, furfuryl acrylate, furfuryl methacrylate, and methylthioethylacrylamide.

In another embodiment, one or more hydrophilic monomers comprise from about 30% to about 65% by weight of the copolymer, or from about 35% to about 65% by weight of the copolymer or from about 40% to about 65% by weight of the copolymer, or from about 45% to about 65% by weight of the copolymer, or from about 50% to about 65% by weight of the copolymer, or from about 55% to about 65% by weight of the copolymer or from about 60% to about 65% by weight of the copolymer.

In yet another embodiment, one or more hydrophilic monomers comprise from about 35% to about 55% by weight of the copolymer, or from about 40% to about 55% by weight of the copolymer, or from about 45% to about 55% by weight of the copolymer, or from about 50% to about 55% by weight of the copolymer.

In yet another embodiment, one or more hydrophilic monomers comprise from about 90% to about 97% by weight of the copolymer, or from about 92% to about 97% by weight of the copolymer, or from about 94% to about 97% by weight of the copolymer, or from about 95% to about 97% by weight of the copolymer.

### Hydrophobic Monomer

Suitable hydrophobic monomers (i.e., monomers whose homopolymers are hydrophobic in accordance with this invention) include, but are not limited to, Lauryl methacrylate, lauryl acrylate, 2-ethylhexyl acrylate, 2-ethylhexyl methacrylate, n-decyl acrylate, n-decyl methacrylate, hexyl acrylate, hexyl metacrylate, stearyl acrylate, stearyl methacrylate, isodecyl acrylate, isodecyl methacrylate, isobornyl acrylate, isobornyl methacrylate, vinyl laurate, vinyl stearate, 1-hexadecyl acrylate, 1-hexadecyl methacrylate, n-myristyl acrylate, n-myristyl methacryalte, n-dodecyl methacrylamide, butyl acrylate, n-butyl methacrylate, isooctyl acrylate, isotridecyl acrylate, isooctyl methacrylate, and isotridecyl methacrylate.

In another embodiment, one or more hydrophobic monomers comprise from about 40% to about 70% by weight of the copolymer, or from about 45% to about 70% by weight of the copolymer or from about 45% to about 70% by weight of the copolymer, or from about 50% to about 70% by weight of the copolymer, or from about 55% to about 70% by weight of the copolymer, or from about 60% to about 70% by weight of the copolymer or from about 65% to about 70% by weight of the copolymer.

In yet another embodiment, one or more hydrophobic monomers comprise from about 45% to about 65% by weight of the copolymer, or from about 50% to about 65% by weight of the copolymer, or from about 55% to about 65% by weight of the copolymer, or from about 60% to about 65% by weight of the copolymer.

### Cross-Linker

Suitable crosslinkers include, for example, but are not limited to, ethylene glycol dimethacrylate (EGDMDA), diethylene glycol dimethacrylate, and triethylene glycol dimethacrylate and poly (ethylene glycol) dimethacrylate wherein ethylene glycol dimethacrylate is preferred. Suitable initiators include, for example, but are not limited to, azobis(isobutyronitrile), 2,2′-azobis(2,4-dimethylvaleronitdle), 2,2′-azobis (methylbutyronitrile), 1,1′-azobis(cyanocyclohexane), di-t-butyl peroxide, dicumyl peroxide, t-butylcumyl peroxide, 2,5-dimethyl-2,5-bis(2-ethylhexanoyl peroxy)hexane, t-butyl peroxyneodecanote, t-butyl peroxy 2-ethylhexanoate, di(4-t-butyl cyclohexyl) peroxydicarbonate, t-butyl peroxypivalate, decanoyl peroxide, lauroyl peroxide, benzoyl peroxide, 2,4-pentanedione peroxide, di(n-propyl) peroxydicarbonate, t-amyl peroxyneodecanoate and t-butyl peroxyacetate wherein 2,2′-azobis(isobutyronitrile) is preferred.

In one embodiment, the crosslinker may present from about 0.1% to about 10% by weight of the composition or from about 0.3% to about 10% by weight of the composition or from about 0.5% to about 10% by weight of the composition or from about 1% to about 10% by weight of the composition or from about 2% to about 10% by weight of the composition or from about 3% to about 10% by weight of the composition or from about 4% to about 10% by weight of the composition or from about 5% to about 10% by weight of the composition or from about 6% to about 10% by weight of the composition.

In one embodiment, a crosslinker may present from about 1% to about 5% by weight of the composition or from about 2% to about 5% by weight of the composition or from about 3% to about 5% by weight of the composition or from about 4% to about 5% by weight of the composition.

### Ultraviolet Light Absorber

Suitable ultraviolet light absorbers include for example but are not limited to beta-(4-benzotriazoyl-3-hydroxyphenoxy) ethyl acrylate, 4-(2-acryloxyethoxy)-2-hydroxybenzophenone, 4-methacryloxy-2-hydroxybenzo-phenone, 2-(2′-methacryloxy-5′-methylphenyl) benzotriazole, 2-(2′-hydroxy-5′-methacryoxyethylphenyl)-2H-benzo-triazole, 2-[3′-tert-Butyl-T-hydroxy-5′-(3″-methacryloyloxypropyl)phenyl]-5-chloro-benzotriazole, 2-(3′-tert-Butyl-5′-[3′-dimethylvinyisilylpropoxy)-2′-hydro-xyphenyl]-5-methoxybenzotriazole, 2-(3′-Allyl-2′-hydroxy-5′-methylphenyl) benzo-triazole, 2-[3′-tert-Butyl-2′-hydroxy-5′-(3″-methacryloyloxypropoxy) phenyl]-5-methoxybenzotriazole, and 2-[3′-tert-Butyl-2′-hydroxy-5′-(3″-methacryloyloxypropoxy) phenyl]-5-chlorobenzo-triazole wherein beta-(4-benzotriazoyl-3-hydroxyphen-oxy)ethyl acrylate is the preferred ultraviolet light absorber.

A UV absorber optionally may be added to the copolymer compositions. A novel, preferred, UV/blue light absorber, i.e., vinyl anthracene, may be added to the copolymer compositions. Conventional U.V. absorbers such as a vinyl benzophenone or a vinyl benzotriazole also may be used.

In another embodiment, a UV absorber may present from about 0.1% to about 5% by weight of the composition or from about 0.2% to about 5% by weight of the composition or from about 0.4% to about 5% by weight of the composition or from about 0.6% to about 5% by weight of the composition or from about 0.8% to about 5% by weight of the composition or from about 1% to about 5% by weight of the composition or from about 1.5% to about 5% by weight of the composition or from about 2% to about 5% by weight of the composition or from about 3% to about 5% by weight of the composition or from about 4% to about 5% by weight of the composition.

### Monomeric Dye

A monomeric dye capable of copolymerizing with the hydrophobic and the hydrophilic monomers optionally may be added to the copolymer to attenuate specific wavelengths of light. Such dyes include but are not limited to those containing vinyl groups and are capable of absorbing violet, blue, red, and green light in the range of 400-700 nm.

Examples of such monomeric dyes include but are not limited to:

Disperse Red 13 acrylate,

Disperse Orange 3 acrylamide

Disperse Orange 3 methacrylamide

Disperse Red 1 methacrylate

Disperse Red 1 acrylate

Disperse Red 13 methacrylate

Disperse yellow 7 acrylate

Disperse yellow 7 methacrylate

Ethyl trans-α-cyano-3-indoleacrylate

[(S)-(−)-1-(4-Nitrophenyl)-2-pyrrolidinemethyl]acrylate

### Methods of Manufacturing

The disclosure provides methods of manufacturing a bioactive polymer. These methods may include (1) selecting a bioactive agent; (2) conjugating the bioactive agent to a moiety to produce a bioactive monomer; and (3) performing a copolymerization reaction using as reactants the bioactive monomer and one or more other monomers each lacking the bioactive agent. The moiety of the bioactive monomer may react directly with at least one of the other monomers in the copolymerization reaction.

The methods also may include (1) selecting a bioactive agent; (2) conjugating the bioactive agent to a first alkenyl moiety to produce a bioactive monomer; and (3) performing an alkenyl-based copolymerization reaction to produce the bioactive polymer. The reactants may include at least the bioactive monomer and one or more other monomers each including an alkenyl moiety and lacking the bioactive agent.

The bioactive polymer may include (1) a bioactive monomer constituent including a bioactive agent; and (2) one or more other monomer constituents each lacking the bioactive agent. The bioactive polymer may include a backbone that covalently links the bioactive monomer constituent to each of the one or more other monomer constituents. Each unit of the bioactive monomer constituent may contribute at least two carbon atoms to the backbone, and each unit of each monomer constituent of the one or more other monomer constituents may contribute at least two carbon atoms (e.g., exactly two carbon atoms to the backbone.

The compositions and methods disclosed herein can be further described by the following paragraphs:

A1. A method of manufacturing a bioactive polymer, the method comprising:

selecting a bioactive agent;

conjugating the bioactive agent to a moiety to produce a bioactive monomer; and

performing a copolymerization reaction using as reactants the bioactive monomer and one or more other monomers each lacking the bioactive agent, and wherein the moiety of the bioactive monomer reacts directly with at least one of the other monomers in the copolymerization reaction.

A2. The method of claim A1, wherein performing produces an acrylic polymer or a silicone polymer.

A3. The method of claim A1 or A2, wherein performing produces a matrix for an ophthalmic lens.

A4. The method of any of claims A3, wherein the matrix is foldable without breaking, tearing, or cutting the matrix.

A5. The method of any of claims A1 to A4, wherein the bioactive agent includes a protein, a protein fragment, a polypeptide of at least 50 amino acids, a peptide of 2-49 amino acids, a proteoglycan, a polysaccharide, an antibacterial agent, and/or a therapeutic agent.

A6. The method of claim A5, wherein the bioactive agent includes an extracellular matrix protein or at least a sequence of 5, 10, 25, 50, or 100 amino acids of the extracellular matrix protein.

A7. The method of claim A6, wherein the extracellular matrix protein is collagen, elastin, keratin, vimentin, fibronectin, or laminin.

B1. A method of manufacturing a bioactive polymer, the method comprising:

selecting a bioactive agent;

conjugating the bioactive agent to a first alkenyl moiety to produce a bioactive monomer; and

performing an alkenyl-based copolymerization reaction to produce the bioactive polymer, using as reactants at least the bioactive monomer and one or more other monomers each including an alkenyl moiety and lacking the bioactive agent.

B2. The method of claim B1, wherein the bioactive agent includes at least one of a protein, a peptide, a polysaccharide, an antibacterial agent, and a therapeutic agent.

B3. The method of claim B1 or B2, wherein at least one of the one or more other monomers is an acrylic compound.

B4. The method of claim B3, wherein the acrylic compound is independently acrylic acid or an alpha/beta-substituted acrylic acid, an acrylate ester or an alpha/beta-substituted acrylate ester, or an acrylic amide or an alpha/beta-substituted acrylic amide.

B5. The method of any of claims B1 to B4, wherein at least one monomer of the one or more other monomers includes a methacrylate compound, a methyl methacrylate compound, a hydroxyethyl acrylate compound, or a hydroxyethyl methacrylate compound.

B6. The method of any of claims B1 to B5, wherein the one or more other monomers include a hydrophilic monomer and a hydrophobic monomer.

B7. The method of claim B6, wherein the hydrophilic monomer has a hydroxyalkyl group bonded directly to an oxygen atom of an ester functional group.

B8. The method of claim B6 or B7, wherein the hydrophobic monomer has an alkyl group bonded directly to an oxygen atom of an ester functional group.

B9. The method of any of claims B1 to B8, wherein the bioactive polymer includes an acrylic polymer.

B10. The method of any of claims B1 to B9, wherein conjugating includes conjugating the bioactive agent to the first alkenyl moiety via at least one linkage selected from an amide, amino, ester, ether, carbonyl, organic acid anhydride, and organosulfur linkage.

B11. The method of any of claims B1 to B10, wherein the first alkenyl moiety is provided by an acrylic compound.

B12. The method of claim B11, wherein the acrylic compound is acrylic acid or an alpha/beta-substituted acrylic acid.

B13. The method of claim B12, wherein the acrylic compound is methacrylic acid.

B14. The method of any of claims B1 to B13, wherein conjugating includes reacting an amine of the bioactive agent with a carboxyl group of the first alkenyl moiety.

B15. The method of claim B14, wherein conjugating includes activating the carboxyl group using 1-ethyl-3-(3-(dimethylamino)propyl) carbodiimide (EDC) and/or N-hydroxysuccinimide (NHS).

B16. The method of claim B14, wherein the amine is provided by one or more lysine residues of the bioactive agent.

B17. The method of any of claims B1 to B16, wherein the one or more other monomers include a crosslinking monomer having at least two alkenyl groups per molecule.

B18. The method of claim B17, wherein the crosslinking monomer is ethylene glycol dimethacrylate.

B19. The method of claim any of claims B1 to B18, wherein the one or more other monomers include a UV-blocking monomer.

B20. The method of claim B19, wherein the UV-blocking monomer is vinyl benzotriazole or vinyl benzophenone.

B21. The method of any of claims B1 to B20, wherein the one or more other monomers include a blue light blocking chromophore.

B22. The method of any of claims B1 to B21, wherein the blue light blocking chromophore includes vinyl anthracene and/or Disperse Orange 3 methacrylamide (2-methyl-N-[4-[(4-nitrophenyl)diazenyl]phenyl]prop-2-enamide).

B23. The method of any of claims B1 to B22, wherein performing is performed in a reaction mixture including a free radical initiator.

B24. The method of any of claims B1, wherein the bioactive polymer has a refractive index of 1.4-1.6 or 1.45-1.55.

B25. The method of any of claims B1 to B24, further comprising hydrating the bioactive polymer.

B26. The method of claim B25, wherein the hydrated bioactive polymer has an equilibrium water content of 10-50% or 15-40%.

B27. The method of any of claims B1 B26, wherein performing is conducted inside a mold.

B28. The method of claim B27, wherein performing includes heating the mold to at least 50, 60, 70, 80, or 100 degrees Celsius.

B29. The method of claim B27 or B28, wherein the mold is shaped to produce an ophthalmic lens.

B30. The method of claim B29, wherein the ophthalmic lens is an intraocular lens or a contact lens.

B31. The method of any of claims B1 to B30, wherein the bioactive agent includes a protein, a protein fragment, a polypeptide of at least 50 amino acids, a peptide of 2-49 amino acids, a proteoglycan, a polysaccharide, an antibacterial agent, and/or a therapeutic agent.

B32. The method of claim B31, wherein the bioactive agent includes an extracellular matrix protein or at least a sequence of 5, 10, 25, 50, or 100 amino acids of the extracellular matrix protein.

B33. The method of claim B32, wherein the extracellular matrix protein is collagen, elastin, keratin, vimentin, fibronectin, or laminin.

B34. The method of any of claims B1 to B33, wherein bioactive polymer is foldable without substantially breaking, tearing, or cutting a matrix of the bioactive polymer.

B35. The method of any of claims B1 to B34, wherein conjugating includes conjugating two or more copies of the first alkenyl moiety to the bioactive agent, such that the bioactive monomer functions as a crosslinking monomer in the copolymerization reaction.

B36. The method of any of claims B1 to B35, wherein the bioactive monomer has a molecular weight that is at least 10, 50, 100, 500, or 1000 times a molecular weight of at least one of the one or more other monomers.

B37. A bioactive polymer produced by the method of any of claims B1 to B36.

B38. An ophthalmic lens including the bioactive polymer of claim B37.

C1. A bioactive polymer, comprising:

a bioactive monomer constituent including a bioactive agent; and

one or more other monomer constituents each lacking the bioactive agent; and

wherein the bioactive polymer includes a backbone that covalently links the bioactive monomer constituent to each of the one or more other monomer constituents, and wherein each unit of the bioactive monomer constituent contributes at least two carbon atoms to the backbone and each unit of each monomer constituent of the one or more other monomer constituents contributes exactly two carbon atoms to the backbone.

C2. The bioactive polymer of claim C1, wherein units of the bioactive monomer constituent are interspersed with units of the one or more other monomer constituents along the backbone.

C3. The bioactive polymer of claim C1 or C2, wherein the one or more other monomer constituents form a bulk of the bioactive copolymer.

C4. The bioactive polymer of claim C3, wherein the bulk of the bioactive polymer is at least 80%, 90%, 95%, or 98% of the bioactive polymer by mass or number of units.

C5. The bioactive polymer of any of claims C1 to C4, wherein the bioactive polymer includes an acrylic polymer.

C6. The bioactive polymer of any claim C1, wherein the one or more other monomers includes a UV-blocking monomer, a crosslinking monomer, and/or a blue light blocking monomer.

### EXAMPLES

The following examples illustrate selected aspects of the present disclosure. They are not intended to limit or define the entire scope of this invention.

### Example 1: Protein Vinyl-Functionalization

This example describes an exemplary method for protein vinyl-functionalization. The free amines of lysine residues are reacted with vinyl groups to create vinyl-protein (VP) (i.e., vinyl collagen in Table 1 and vinyl gelatin, vinyl fibronectin, vinyl laminin, vinyl heparin, and vinyl hyaluronic acid in Table 2). The carboxyl group of methacrylic acid is activated with 1-ethyl-3-(3-(dimethylamino)propyl) carbodiimide (EDC) and N-hydroxysuccinimide (NHS) in MES buffer. This mixture is added to the protein at 3.75 mg/mL in 0.02 N acetic acid to form the final product. The product is dialyzed and lyophilized. Collagen type I was used as a representative bioactive protein for the polymers tested in Table 1.

### Example 2: Methods for Preparing Polymers

This example describes exemplary methods for preparing bioactive polymers using the vinyl-protein of Example 1. Various polymer formulations and results obtained therewith are listed in Table 1.

Various copolymers are prepared by mixing the following ingredients under reduced pressure: (1) comonomers (e.g., VP, HEA, and MMA, or VP, HEMA, and LM), (2) a crosslinker (e.g., EGDM), and (3) a polymerizable UV blocking agent. Vinyl benzotriazole or vinyl benzophenone at a total concentration of 0.1-5.0% by weight is utilized as a UV blocking agent. Blue light blocking chromophores may also be added at a concentration of 0.01% by weight. To initiate polymerization, a free radical initiator (e.g., AIBN) is employed at a concentration of 0.05-1.0% by weight. The monomer solution is mixed in a glass flask using a magnetic stir bar for 30 minutes. A small amount of formic acid can be added if turbidity develops in the solution. The solution is then filtered through a 0.2 micron filter and injected into a sheet mold comprising two glass plates held together with spring clips and separated by a plastic gasket. The mold is then placed in a water bath for 4 hours at 60° C. followed by 6 hours at 70° C.

Table 1 provides the composition of various bioactive polymers with vinyl collagen along with associated Refractive Index and Equilibrium water content.

Table 2 provides the compositions of various bioactive polymers with the bioactive agent being either gelatin, fibronectin, laminin, heparin, and hyaluronic acid. Polymers 1 and 2 in Table 2 showed excellent results.

TABLE 1
Results obtained for various polymer formulations with vinyl collagen.

| | Polymer | | | | | | | |
|---|---|---|---|---|---|---|---|---|
| Monomer by weight | 1 | 2 | 3 | 4 | 5 | 6 | 7 | 8 |
| Vinyl Collagen (VP) | 0.002 | 0.005 | 0.01 | 0.02 | 0.002 | 0.005 | 0.01 | 0.02 |
| Hydroxyethyl acrylate | | | | | 0.45 | 0.55 | 0.3 | 0.53 |
| (HEA) | | | | | | | | |
| Hydroxyethyl | 0.45 | 0.55 | 0.3 | 0.53 | | | | |
| methacrylate (HEMA) | | | | | | | | |
| Lauryl Methacrylate | 0 | 0.415 | 0.66 | 0.42 | | | | |
| (LM) | | | | | | | | |
| Methylmethacrylate | 0.518 | | | | 0.518 | 0.415 | 0.66 | 0.42 |
| (MMA) | | | | | | | | |
| Ethyleneglycol | 0.03 | 0.03 | 0.03 | 0.03 | 0.03 | 0.03 | 0.03 | 0.03 |
| dimethcarylate | | | | | | | | |
| (EGDM) | | | | | | | | |
| RI | 1.503 | 1.484 | 1.467 | 1.483 | 1.487 | 1.485 | 1.489 | 1.485 |
| EWC | 28 | 34 | 19 | 33 | 28 | 34 | 19 | 33 |

RI: Refractive Index
EWC: equilibrium water content
0.3% by weight of MEB was used in all copolymer compositions.
MEB: 2-(2′-Methacryloxy-5′methylphenyl)benzotriazole

TABLE 2
Results obtained for various polymer formulations with vinyl gelatin.

| | Polymer | | | | | | | | | |
|---|---|---|---|---|---|---|---|---|---|---|
| Monomer by weight | 1 | 2 | 3 | 4 | 5 | 6 | 7 | 8 | 9 | 10 |
| Vinyl Gelatin | 0.002 | 0.002 | 0.002 | 0.002 | 0.002 | 0.002 | | | | |
| Vinyl fibronectin | | | | | | | 0.002 | | | |
| Vinyl laminin | | | | | | | | 0.002 | | |
| Vinyl heparin | | | | | | | | | 0.002 | |
| Vinyl hyaluronic acid | | | | | | | | | | 0.002 |
| Hydroxyethyl methacrylate | 0.9585 | 0.9580 | 0.9600 | 0.9530 | 0.9440 | 0.9340 | 0.9585 | 0.9585 | 0.9585 | 0.9585 |
| (HEMA) | | | | | | | | | | |
| Ethyleneglycol | 0.0015 | 0.0020 | 0.0028 | 0.0100 | 0.0200 | 0.0300 | 0.0015 | 0.0015 | 0.0015 | 0.0015 |
| dimethcarylate (EGDM) | | | | | | | | | | |
| MHBPH | 0.0380 | 0.0380 | 0.0350 | 0.0350 | 0.0350 | 0.0350 | 0.0380 | 0.0380 | 0.0380 | 0.0380 |
| RI (hydrated) | 1.446 | 1.447 | 1.451 | 1.452 | 1.456 | 1.458 | 1.448 | 1.442 | 1.446 | 1.444 |
| EWC | 38 | 36 | 31 | 29 | 28 | 27 | 37 | 38 | 36 | 36 |

RI: Refractive Index
EWC: equilibrium water content
MEB: 4-methacryloxy-2-hydroxybenzophenone

The post-curing steps are carried out as follows. 100° C. in gravity oven for 24 hours followed by 120° C. in a vacuum oven for another 24 hours. A clear polymer sheet is obtained. A disk that is 1 cm in diameter and 2 mm in thickness is cut from the sheet and hydrated. The equilibrium water content (EWC) is calculated based on the hydrated weight and dry weight.

### Example 3: Molding Method

This example describes an exemplary molding method. A monomer mixture is heated to 80° C. for about 30 minutes or until the viscosity is increased to 1000 Cps. The monomer mixture is then quenched using an ice bath. The cooled monomer solution is injected into intraocular lens (IOL) molds, and the molds are transferred to a programmable oven having the following cycle:

1. 60° C. for 4 hours

2. 80° C. for 8 hours

3. 100° C. for 4 hours

4. 120° C. for 4 hours in vacuo

The IOLs are demolded and stored in glass vials for further testing.

While the invention has been described through the above examples and features, those of ordinary skill in the art will understand that a wide variety of modifications, combinations, and variations of the examples and features may be made without departing from the inventive concepts herein disclosed. Moreover, the invention should not be viewed as being limited to any specific purposes or embodiments described herein, but rather should be viewed as being applicable to accomplish a wide variety of purposes beyond those described herein. This disclosure describes some examples of the present technology, in which only some of the possible examples are shown. Other aspects can, however, be embodied in many different forms and should not be construed as limited to the examples set forth herein even if not expressly exemplified in combination. Rather, these examples were provided so that this disclosure is thorough and complete and fully conveys the scope of the possible examples to those skilled in the art.

What is claimed is:

1. A method of manufacturing a bioactive polymer, the method comprising:
conjugating a bioactive agent to a moiety to produce a bioactive monomer; and
performing a copolymerization reaction using as reactants the bioactive monomer and one or more other monomers each lacking the bioactive agent, and wherein the moiety of the bioactive monomer reacts directly with at least one of the other monomers in the copolymerization reaction.

2. The method of claim 1, wherein performing produces an acrylic polymer or a silicone polymer.

3. The method of claim 1, wherein the bioactive agent includes a protein, a protein fragment, a polypeptide of at least 50 amino acids, a peptide of 2-49 amino acids, a proteoglycan, a polysaccharide, an antibacterial agent, and/or a therapeutic agent.

4. The method of claim 1, wherein the bioactive agent includes an extracellular matrix protein or at least a sequence of 5, 10, 25, 50, or 100 amino acids of the extracellular matrix protein.

5. The method of claim 4, wherein the extracellular matrix protein is collagen, elastin, keratin, vimentin, fibronectin, or laminin.

6. The method of claim 1, wherein at least one monomer of the one or more other monomers includes a methacrylate compound, a methyl methacrylate compound, a hydroxyethyl acrylate compound, or a hydroxyethyl methacrylate compound.

7. The method of claim 1, wherein the one or more other monomers include a hydrophilic monomer and a hydrophobic monomer.

8. The method of claim 7, wherein the hydrophilic monomer has a hydroxyalkyl group bonded directly to an oxygen atom of an ester functional group.

9. The method of claim 7, wherein the hydrophobic monomer has an alkyl group bonded directly to an oxygen atom of an ester functional group.

10. A method of manufacturing a bioactive polymer comprising:
conjugating a bioactive agent to a first alkenyl moiety to produce a bioactive monomer; and
performing an alkenyl-based copolymerization reaction to produce the bioactive polymer, using as reactants at least the bioactive monomer and one or more other monomers each including an alkenyl moiety and lacking the bioactive agent.

11. The method of claim 10, wherein the bioactive agent is selected from the group consisting of: at least one of a protein, a protein fragment, a peptide, a polysaccharide, a polypeptide of at least 50 amino acids, a proteoglycan, an extracellular matrix protein, an antibacterial agent, and a therapeutic agent.

12. The method of claim 10, wherein at least one of the one or more other monomers is an acrylic compound.

13. The method of claim 12, wherein the acrylic compound is independently acrylic acid or an alpha/beta-substituted acrylic acid, an acrylate ester or an alpha/beta-substituted acrylate ester, or an acrylic amide or an alpha/beta-substituted acrylic amide.

14. The method of claim 10, wherein at least one monomer of the one or more other monomers includes a methacrylate compound, a methyl methacrylate compound, a hydroxyethyl acrylate compound, or a hydroxyethyl methacrylate compound.

15. The method of claim 10, wherein the one or more other monomers include a hydrophilic monomer and a hydrophobic monomer.

16. The method of claim 15, wherein the hydrophilic monomer has a hydroxyalkyl group bonded directly to an oxygen atom of an ester functional group.

17. The method of claim 15, wherein the hydrophobic monomer has an alkyl group bonded directly to an oxygen atom of an ester functional group.

18. The method of claim 10, wherein the bioactive polymer has a refractive index of 1.4-1.6 or 1.45-1.55.

19. The method of claim 10, further comprising hydrating the bioactive polymer, wherein the hydrated bioactive polymer has an equilibrium water content of 10-50% or 15-40%.

20. A bioactive polymer produced by the method of claim 10.
